(19) United States
(12) Patent Application Publication
(10) Pub. No.: US 2007/0050043 A1
(43) Pub. Date: Mar. 1, 2007

(54) VERTEBROPLASTY COMPOSITIONS & METHODS

(75) Inventor: JAN ALBERT de VRIES, ZELHEM (NL)

Correspondence Address: TROUTMAN SANDERS LLP, 600 PEACHTREE STREET , NE, ATLANTA, GA, 30308, US

(73) Assignee: BROOCKEVILLE CORPORATION, N.V., WILLEMSTAD (AN)

(21) Appl. No.: 11/553,403

(22) Filed: Oct. 26, 2006

Related U.S. Application Data
(63) Continuation of application No. 11/078,146, filed on Mar. 11, 2005.
(60) Provisional application No. 60/553,100, filed on Mar. 15, 2004.

(51) Int. Cl. A61F 2/28

(52) U.S. Cl. 623/23.61

(57) ABSTRACT

Vertebroplasty compositions and methods are provided. By providing an elastic form stable material which is capable of being delivered directly to a specific desired location within a living creature and provides increased strength and rigidity to the injected location, disorders of the vertebrae of a living creature, for example disorders derived from osteoporosis, are able to be effectively treated. In a method embodiment, elastic form stable material is injected directly into a vertebral body to reinforce and strengthen the vertebral body. Other embodiments are also claimed and described.

### CROSS-REFERENCE TO RELATED APPLICATIONS

This application is a continuation of United States Non-Provisional application Ser. No. 11/078,146, filed 11 Mar. 2005, which claims the priority and benefit of U.S. Provisional Application No. 60/553,100, filed 15 Mar. 2004, and both applications are incorporated herein by reference as if fully set forth below in their entirety.

### TECHNICAL FIELD

The various embodiments of the present invention relate generally to vertebroplasty compositions and methods and, more specifically, to treating vertebral bodies in living creatures, in particular, human beings and also compositions suitable for use in such methods and preparation and use of such compositions.

### BACKGROUND

Percutaneous vertebroplasty comprises the injection of bone filler into a vertebral body to be treated via a percutaneous route, usually under X-Ray guidance, such as lateral projection fluoroscopy. The bone filler is injected as a paste or semi-liquid from a suitable gun or injection system via a needle, that has been passed into the vertebral body. Vertebroplasty is usually applied to patients that suffer from osteoporotic fractures, malignant metastatic disease, and benign tumors of the bone. The bone filler, once injected, should provide a reinforcement to and improved compressive strength of the vertebral body. In addition to these reinforcing and strengthening properties, it is desirable that the starting bone filler composition has a viscosity that allows it to flow into the fracture planes.

At present almost all conventional bone fillers are based on polymethyl methacrylate. This type of bone filler is prepared from a mixture of methyl methacrylate powder and a suitable radiopaque agent (opacifier) to which a liquid monomer is added. Then the resulting filler should be injected within a limited period of time, e.g. 5 to 10 minutes before the filler starts to thicken and becomes unworkable. While the filler is being injected, continuous X-Ray imaging is performed to watch carefully whether the filler is deposited in the appropriate position and proportion, typically some cubic centimeters, within the vertebral body. After injection, the filler is cured within the vertebral body to a hard material.

While serving their respective purposes, conventional polymethyl methacrylate based filler materials do have several drawbacks. For example, due to its hardness and strength, the cured filler can easily distort the brittle bone material of a vertebral body into which it has been injected. In addition, the hard and stiff filler material can act as an abrasive within bone pores and migrate through softer vertebral bone. There is also a risk of leakage from a vertebral body, thereby cutting through or exerting pressure on tissue and nerves. Also, the polymethyl methacrylate material can be carried into the venous system as such or as free monomer residue leading to a potential occurrence of fatal hypertension episodes. The polymethyl methacrylate conventional material also can cause substantial exothermic reaction (i.e., substantial heat generation) to occur during the curing process leading potentially to bone necrosis.

Accordingly, there is a need for vertebroplasty compositions and methods that provide and enable safe vertebroplasty conditions that do not jeopardize the brittle bone material of a vertebral body. There is also a need for such compositions and methods capable of enabling easily prepared flexible and viscous bone filler materials. It is to the provision of such vertebroplasty compositions and methods that the various embodiments of the present invention are directed.

### BRIEF SUMMARY

Therefore, it is an object of embodiments of the present invention to provide filler material and a method for using the filler material in a vertebral body, or other similar application, which is easily prepared and delivered to the vertebral body while also providing the desired reinforcing and strengthening properties.

Another object of embodiments of the present invention is to provide filler material and a method for using the filler material in a vertebral body, or similar application which is flexible and viscous to provide flow ability throughout the vertebral body both during its application and after curing to enable effective fixation of the brittle bone structure and absorb the forces applied to it. Additional objects and features of embodiments of the present invention are discussed below and others will become apparent to those skilled in the art.

By employing various embodiments of the present invention drawbacks of conventional materials can be eliminated. Thus, various embodiments of the present invention provide vertebroplasty methods to treat disordered vertebral bodies in a living creature, in particular a human being, as well as treating other similar areas of an individual. Additional embodiments also provide compositions for formulating improved bone filler materials. Also method embodiments can comprise injecting a curable bone filler composition in a vertebral body and the curable bone filler can comprise an elastic form stable material.

According to some embodiments of the present invention, a creature such as a human being in need of a vertebroplasty treatment, e.g. derived from osteoporosis, can be treated by reinforcing and strengthening a particular vertebral body or bodies. Indeed, a bone filler can be injected into a selected vertebral body and the bone filler can be an elastic form stable material. Due to its properties with respect to elasticity and dimensional stability, forces exerted on and within the brittle vertebral body upon and after injecting are smaller, thereby reducing the risk of breaking or disrupting treated bodies. After curing, this non-resorbable material is preferably a solid but still a flexible mass not likely to migrate through the body that holds its initial position.

Typically, a vertebroplasty method embodiment according to the present invention can comprise several steps. A patient is generally initially examined by CT and/or MR imaging to provide data upon which a decision about a vertebral body to be treated is made. The patient is positioned in a prone position, and skin covering the vertebral body is prepared. The patient is anesthetized usually by locally injecting a suitable anesthetic into the skin underlying fat and into the periosteum of the pedicle to be entered.

Next, a skin incision is made using a scalpel or other surgical instrument. A needle having a suitable gauge is selected and passed down the pedicle into the respective vertebral body. Alternatively, the needle or catheter may be introduced through the posterior-lateral aspect of the vertebral body. If desired, any unwanted leakage can be detected by injecting a suitable X-Ray contrast liquid into the vertebral body and performing a venogram.

Meanwhile, a filler is prepared and then injected into the vertebral body using an injection means such as a needle. Needle position and amount of injected filler material is preferably monitored, for example, by using lateral X-Ray projection fluoroscopy imaging. A suitable opacifier can be added to the bone filler to aid in imaging. The injection is stopped when filling of the vertebral body has been completed to the desired extent. If the filler starts to flow into unwanted locations, injection can also be interrupted.

In some method embodiments according to the present invention elastic form stable material preferably comprises an elastomer-precursor composition which is cured in situ. The elastomer precursor is preferably prepared in advance and then applied to an appropriate vertebral body by suitable equipment. The filler material is substantially less toxic than conventional materials. Thus, the filler material composition according to embodiments of the present invention can be tolerated easily by patients.

Embodiments of the present invention also relate to preparing a composition for treating a disordered vertebral body in a living creature, in particular a human being, comprising an elastic form stable material. Method embodiments can comprise providing starting materials of an elastic form stable material in an appropriate ratio into separate containers (or separated chambers that may be present in one mixing/dispensing device), and packaging thereof.

Other aspects and features of embodiments of the present invention will become apparent to those of ordinary skill in the art, upon reviewing the following description of specific, exemplary embodiments of the present invention in conjunction with the accompanying figure.

### BRIEF DESCRIPTION OF THE DRAWING

For a fuller understanding of the nature and objects of the present invention, reference should be held to the following detailed description taken in connection with the accompanying drawings, and which:

FIG. 1 is a cross-sectional side elevation view depicting three adjacent vertebral bodies, with the composition of the present invention in the process of being used in treating one of the vertebral bodies.

### DETAILED DESCRIPTION OF PREFERRED & ALTERNATIVE EMBODIMENTS

Referring now to the figure, wherein exemplary embodiments of the present invention will be described in detail. Throughout this description, various components or features may be identified having specific values or parameters, however, these items are provided as exemplary embodiments. Indeed, the exemplary embodiments do not limit the various aspects and concepts of the present invention as many comparable parameters, sizes, ranges, and/or values may be implemented.

According to a preferred embodiment of the present invention, the vertebroplasty composition comprises a curable elastomer-precursor composition. Commercially available medical grade silicone elastomers are preferred materials for use as polymer precursor in this composition. A more preferred material is poly (dimethyl siloxane) such as hydroxyl-end-blocked poly (dimethyl siloxane). Such silicone elastomers of medical grade as pourable, multi-component silicones are commercially available.

For these types of elastomers, propyl orthosilicate is a useful cross-linking agent. Fillers and diluents (medicinal fluids such as known under the trade name Dimeticonum) in order to reduce viscosity may be added as needed. It is preferred that a well flowable composition is used, so that even small cracks are filled with the composition.

In a preferred formulation, an initiator like tin (II) octoate initiates the polymerization reaction with splitting of propanol. The reaction proceeds without the generation of sensible heat, which is also beneficial to the patient treated. Silphenylene polymer can be used in a similar way. To be able to trace the position of the cured applied composition by X-Ray monitoring, a preferred embodiment of the composition of the present invention, comprises a radiopaque material, such as silver powder, barium sulfate, bismuth trioxide, zirconium dioxide, tantalum or titanium powders or fibers, calcium sulfate, calcium phosphate, hydroxyapetite, tri calcium phosphate, and other medically appropriate opacifier agents. Silver powder can be used as a radiopaque material, since smaller proportions are needed in the composition and the material also provides a material anti-microbial activity.

A composition as explained above is known, per se, as a material suitable for the non-surgical, irreversible sterilization of females. For example, U.S. Pat. No. 4,245,623, which is hereby incorporated by reference, teaches such a composition. In this known sterilization method, the composition is injected in the oviduct portion adjacent the uterus, where it forms in situ a block or plug in the oviduct, thereby preventing the passage of ovum from the ovaries to the uterus and sperm from entering the oviduct and thus conception. However, the embodiments of the present invention are patentably distinguished over this patent.

A preferred composition for use in the method according to the invention comprises about 60-85% by weight poly (dimethyl siloxane), about 2-5% cross-linking agent, a diluent in the range of 10-20%, and about 10-20% radiopaque powder.

In a method embodiment according to the present invention a composition is advantageously prepared in advance in a mixing-dispensing device. Such a device, wherein the function of mixing the components is combined with the function of dispensing the thus prepared mixture, is known per se, e.g. from the above U.S. patent, the content of which is incorporated in its entirety by reference. In addition, other mixing and dispensing systems known in the art.

One of these alternate, two-component mixing and dispensing devices is fully disclosed in pending U.S. patent application Ser. No. 11/000,578, filed Nov. 30, 2004, entitled Two Component Mixing and Dispensing Device. If desired, the composition and method of the present invention may be employed using this device and the pertinent disclosure of this pending patent application is incorporated herein by reference.

As indicated above, embodiments of the present invention relate to methods of preparing an injection composition for injection into a disordered vertebral body of a living creature, in particular a human being. The composition can comprise an elastic form stable material. The above-mentioned preferred features of the treatment method according to the invention are similarly applicable to the preparation method according to embodiments of the present invention.

Advantageously the injection composition can be packaged as a kit of parts. The kit can comprise a first container filled with an elastomer precursor and optionally a diluent, and a second container filled with a cross-linking agent for this elastomer precursor. More preferably, the composition is packaged in a mixing-dispensing device, comprising such containers with a temporary seal between the containers. In addition, one of these containers is provided with a stirrer which can be operated manually or powered by an external source. An example of such a device is also known from the above-mentioned application, U.S. patent application Ser. No. 11/000,578. Devices of this type can be used for injecting the thus prepared precursor composition by connecting a suitable flexible tube to the container acting as a mixing chamber and providing an appropriate needle at the other end of the tube.

In accordance with embodiments of the present invention a flowable composition is prepared from the various components. Preferably prepared in a combined mixing-dispensing device as explained above, and then immediately used. To assist in accurately positioning the deposit of the composition, the needle employed typically has an open tip, and may have one or more exits on the side face directly adjacent to the tip. Once the vertebral body is filled, the needle can be retracted and the composition is allowed to cure in situ. If necessary, these actions can be repeated.

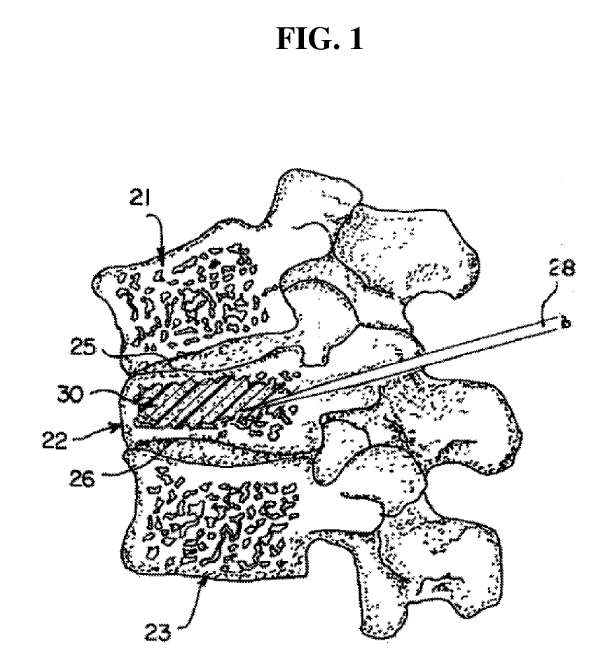

To best understand some exemplary embodiments of this invention, reference is made to FIG. 1, along with the following disclosure. In FIG. 1, three adjacent vertebral bodies 21, 22, and 23 are depicted with varying stages of osteoporosis. As shown, vertebral body 22 has the most serious problem, with end plates 25 and 26 thereof angularly pitched towards each other. To prevent any further deteriorations of vertebral body 22, composition and method embodiments of the present invention can be employed. An elastic form stable, curable bone filler material detailed above is prepared and is readied for delivery to vertebral body 22. Needle 28 is inserted into an individual, with the needle's tip inserted into vertebral body 22. Then, filler material 30 is forced through needle 28 directly into the cavity of vertebral body 22.

Due to the controlled viscosity of filler material 30, filler material 30 is able to freely flow through the bony structure of vertebral body 22, completely filling the cavity thereof. Once the desired area has been completely filled, the flow of filler material 30 is stopped and needle 28 is removed. Filler material 30 is allowed to cure in situ, thereby providing vertebral body 22 with a solid, flexible mass which is able to strengthen and reinforce the vertebral body and prevent further degradation thereof. Further, due to inherent viscosity of filler material 30 after curing, filler material 30 is also able to provide a self-adjusting movement or shifting within the vertebral body 22, providing strength and reinforcement to any area or zone where such reinforcement or strength is needed.

In addition, the durometer of filler material 30, when cured and retained in the enclosed area defined by vertebral body 22, enables filler material 22 to provide inherent strength and rigidity wherever required, while being compressible and eliminating distortion and/or breakage of brittle bone material in the vertebral body. Filler material 30 may be formulated to cure into a particularly desired durometer which may vary over a wide range.

By controlling the durometer of cured filler material 30, a customized result can be achieved wherein filler material 30 is selected and formulated for treating a particular pathology in the optimum manner. Also, filler material 30 is relatively soft and compressible, thereby allowing the vertebral body to interact with the adjacent vertebral bodies and discs by absorbing shocks or impacts, reducing possible damage to the surrounding elements. Although FIG. 1 depicts the addition of filler material 30 to only vertebral body 22, filler material 30 can be delivered to many vertebral bodies. Consequently, if vertebral body 21 and/or 23 were damaged and in needing repair, the same process could be employed to deliver filler material 30 to these vertebral bodies, or any other area in need of repair.

The embodiments of the present invention are not limited to the particular formulations, process steps, and materials disclosed herein as such formulations, process steps, and materials may vary somewhat. Moreover, the terminology employed herein is used for the purpose of describing exemplary embodiments only and the terminology is not intended to be limiting since the scope of the various embodiments of the present invention will be limited only by the appended claims and equivalents thereof. For example, temperature and pressure parameters may vary depending on the particular materials used.

Therefore, while embodiments of this invention have been described in detail with particular reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all equivalents.

I claim:

1. A vertebroplasty method to treat a disordered vertebral body comprising: injecting a curable bone filler composition in said vertebral body, wherein the curable bone filler material comprises an elastic form stable material.

2. The method of claim 1, wherein said composition comprises a curable elastomer-precursor composition.

3. The method of claim 1, wherein said composition comprises a silicone elastomer.

4. The method of claim 3, wherein said silicone elastomer comprises poly (dimethyl siloxane).

5. The method of claim 1, wherein said composition comprises a cross-linking agent and a diluent.

6. The method of claim 1, wherein said composition comprises a radiopaque material.

7. The method of claim 6, wherein said radiopaque material comprises one of silver powder, barium sulfate, bismuth trioxide, zirconium dioxide, tantalum or titanium powders or fibers, calcium sulfate, calcium phosphate, hydroxyapetite, tri calcium phosphate, and other medically appropriate opacifier agents.

8. The method of claim 1, wherein the curable bone filler material further comprises:
between about 60% and 85% by weight based upon the weight of the entire composition of poly (dimethyl siloxane);
between about 2% and 5% by weight based upon the weight of the entire composition of the cross-linking agent;
between about 10% and 20% by weight based upon the weight of the entire composition of the diluent; and
between about 10% and 20% by weight based upon the weight of the entire composition of the radiopaque material.

9. The method of claim 1, wherein said composition is prepared in advance in a mixing-dispensing device.

10. The method of claim 1 wherein said composition is delivered to the vertebral body by inserting a needle into the internal cavity of the vertebral body and causing the filler material to flow through the needle into the vertebral body.

11. The method of claim 10, further comprising stopping the flow of the filler material when the vertebral body has been filled with the filler material, and withdrawing the needle from the vertebral body.

12. The method of claim 1, wherein said filler material flexible to move, shift, compress, and or elongate within the vertebral body, thereby providing varying actions or reactions.

13. A method to prepare a composition for injection into a disordered area of a body of a living creature, the composition comprising an elastic form stable material consisting of a curable elastomer-precursor composition and additives intermixed therewith, the composition having reduced toxicity.

14. The method of claim 13, wherein the curable elastomer-precursor composition comprises a silicone elastomer.

15. The method of claim 14, wherein said silicone elastomer is poly (dimethoxy siloxane).

16. The method of claim 13, wherein the additives comprise one of a cross-linking agent, a diluent, and a radiopaque material.

17. The method of claim 13, wherein the composition is a kit of parts, comprising a first container with said silicone elastomer, and a second container with said cross-linking agent.

18. The method of claim 17, wherein said kit of parts comprises a mixing-dispensing device, comprising said container and a temporary seal between the containers, wherein one container is provided with a movable stirrer.

19. The method of claim 13, further comprising thoroughly intermixing:
between about 60% and 85% by weight based upon the weight of the entire composition of poly (dimethyl siloxane);
between about 2% and 5% by weight based upon the weight of the entire composition of the cross-linking agent;
between about 10% and 20% by weight based upon the weight of the entire composition of the diluent; and
between about 10% and 20% by weight based upon the weight of the entire composition of the radiopaque material, thereby forming the desired inject, composition.
